(19) United States
(12) Patent Application Publication
(10) Pub. No.: US 2021/0050084 A1
(43) Pub. Date: Feb. 18, 2021

(54) MEDICAL DEVICE SYSTEM AND RELATED OPERATING METHODS

(71) Applicant: MEDTRONIC MINIMED, INC., Northridge, CA (US)

(72) Inventors: Yuxiang Zhong, Arcadia, CA (US); Huzefa F. Neemuchwala, Simi Valley, CA (US); Pratik Agrawal, Porter Ranch, CA (US); Chantal M. McMahon, Atlanta, GA (US); Nolan Lindeke, San Diego, CA (US); Catherine T. Fogel, Castaic, CA (US); Taly G. Engel, Los Angeles, CA (US)

(21) Appl. No.: 16/541,074

(22) Filed: Aug. 14, 2019

(51) Int. Cl. G16H 20/17; A61M 5/142; A61M 5/172; G16H 10/60

(52) U.S. Cl. CPC G16H 20/17; A61M 5/14244; G16H 20/60; G16H 10/60; A61M 5/1723

(57) ABSTRACT

A health care provider (HCP) assistant system includes at least one processor device and a processor-readable medium that stores executable instructions configurable to cause the at least one processor device to perform a method that collects and maintains medical device user data that includes data associated with a patient under the care of an HCP customer. The method also collects and maintains HCP data that includes data associated with the HCP customer. The collected medical device user data and the collected HCP data is reviewed to automatically generate content to assist the HCP customer in managing treatment of the patient. The generated content is provided to an HCP user system for presentation to the HCP customer.

### TECHNICAL FIELD

Embodiments of the subject matter described herein relate generally to a medical device system. More particularly, embodiments of the subject matter relate to systems and methods that generate therapy recommendations, implement recommended changes to patient therapy, and manage treatment of patients who use medication fluid infusion devices, such as insulin infusion pumps.

### BACKGROUND

Portable medical devices, such as medication infusion devices, are useful for patients that have conditions that must be monitored on a continuous or frequent basis. For example, diabetics are usually required to modify and monitor their daily lifestyle to keep their blood glucose (BG) in balance. Individuals with Type 1 diabetes and some individuals with Type 2 diabetes use insulin to control their BG levels. To do so, diabetics are advised to routinely keep strict schedules, including ingesting timely nutritious meals, partaking in exercise, monitoring BG levels daily, and adjusting and administering insulin dosages accordingly.

The prior art includes a number of fluid infusion devices and insulin pump systems that are designed to deliver accurate and measured doses of insulin via infusion sets (an infusion set delivers the insulin through a small diameter tube that terminates at, e.g., a cannula inserted under the patient's skin). In lieu of a syringe, the patient can simply activate the insulin pump to administer an insulin bolus as needed, for example, in response to the patient's high BG level. A patient can monitor BG levels using a BG meter or measurement device and by using a continuous glucose sensor if so desired.

In practice, many processes and behaviors result in fluctuations in BG levels. Commonly recognized processes and factors impacting BG levels include food, exercise, disease (acute or chronic), medication (insulin, oral, etc.), and stress and sleep patterns, among others. Furthermore, behavioral and environmental factors such as the time of the day, attentiveness to therapy, and insulin pump maintenance, can provide additional quantitative indications of the underlying factors impacting glucose control. Current reporting tools available to diabetes patients and their caregivers are patient-specific in that they collect and analyze data in a way that is intended to address an individual patient's particular glycemic outcome.

Accordingly, it is desirable to have a medical device system and related methodologies that support enhanced and more intelligent reporting to health care providers to enable them to manage treatment of patients in a more efficient and effective manner. Furthermore, other desirable features and characteristics will become apparent from the subsequent detailed description and the appended claims, taken in conjunction with the accompanying drawings and the foregoing technical field and background.

### BRIEF SUMMARY

A medical device system of the type described herein includes: a medical device to provide therapy to a patient under the care of a health care professional (HCP) customer; a computer-implemented HCP assistant system; a database system associated with the HCP assistant system; and a computer-implemented HCP user system. The medical device has a processor device and a communication module that facilitate data communication between the medical device and at least one other component of the medical device system. The HCP assistant system includes a processor device and a communication module that facilitate data communication between the HCP assistant system and at least one other component of the medical device system. The database system collects and maintains medical device user data and HCP data. The medical device user data includes data associated with the patient, and the HCP data includes data associated with the HCP customer. The HCP user system includes a processor device and a communication module that facilitate data communication between the HCP user system and at least one other component of the medical device system. The HCP assistant system is operative to: review the collected medical device user data and the collected HCP data; automatically generate content to assist the HCP customer in managing treatment of the patient, based on the review of the collected medical device user data and the collected HCP data; and provide the generated content to the HCP user system for presentation to the HCP customer.

A computer-implemented HCP assistant system is also disclosed herein. An exemplary embodiment of the HCP assistant system includes at least one processor device and a non-transitory processor-readable medium operatively associated with the at least one processor device. The processor-readable medium has executable instructions configurable to cause the at least one processor device to perform a method that involves: collecting and maintaining medical device user data that includes data associated with a patient under the care of an HCP customer; collecting and maintaining HCP data that includes data associated with the HCP customer; reviewing the collected medical device user data and the collected HCP data; automatically generating content to assist the HCP customer in managing treatment of the patient, based on the review of the collected medical device user data and the collected HCP data; and providing the generated content to an HCP user system for presentation to the HCP customer.

Also disclosed is a method of managing use of a medical device that provides therapy to a patient under the care of a HCP customer. An exemplary embodiment of the method involves: operating a database system associated with a computer-implemented HCP assistant system, the HCP assistant system comprising a processor device and a communication module that facilitate data communication between the HCP assistant system and the medical device; collecting and maintaining medical device user data at the database system, the medical device user data including data associated with the patient and further including data associated with operation of the medical device; collecting and maintaining HCP data at the database system, the HCP data including data associated with the HCP customer and further including data associated with treatment of the patient by the HCP customer; reviewing the collected medical device user data and the collected HCP data; automatically generating content to assist the HCP customer in managing treatment of the patient, based on the review of the collected medical device user data and the collected HCP data; and providing the generated content to an HCP user system for presentation to the HCP customer.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a block diagram representation of an exemplary embodiment of a medical device system;

FIG. 2 is a block diagram that includes various components of the medical device system depicted in FIG. 1;

FIG. 3 is a block diagram of a medical device system that includes an insulin infusion device and associated components;

FIG. 4 is a simplified block diagram representation of an exemplary embodiment of a computer-based or processor-based device suitable for deployment in the medical device system shown in FIG. 1; and

FIG. 5 is a flow chart that illustrates an exemplary embodiment of a medical device management process.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. In certain embodiments, the program or code segments are stored in a tangible processor-readable medium, which may include any medium that can store or transfer information. Examples of a non-transitory and processor-readable medium include an electronic circuit, a semiconductor memory device, a ROM, a flash memory, an erasable ROM (EROM), a floppy diskette, a CD-ROM, an optical disk, a hard disk, or the like.

The following description relates to a medical device system that includes or cooperates with medical devices that are owned or operated by patients, and that includes or cooperates with computer-based or processor-based systems that are owned or operated by health care providers (HCPs) who provide medical care, advice, and/or treatment to the patients. In accordance with the exemplary embodiment mentioned here, the medical device system is designed and utilized as a diabetes patient support system that generates and delivers therapy-related content, advice, guidance, and/or recommendations to end users. Although not limited to any particular use case, the primary end users for the exemplary embodiment described here are HCPs (e.g., healthcare professionals, caregivers, doctors, payors, nurses, etc.) In addition, the medical device system may also generate content that is intended for patients. In certain embodiments, the medical device system may also generate control commands, therapy delivery instructions, and/or device configuration settings that are formatted for execution at the medical devices used to treat the patients. Accordingly, the system described here can be utilized to provide therapy-related content to patients if so desired.

The exemplary implementation and deployment disclosed herein leverages a cloud-based architecture in that most of the processor-intensive tasks are performed by one or more server systems that communicate with remotely located computer-based client devices. The disclosed medical device system obtains and processes patient data for a population of different patients under the care of different physicians. Patient data can originate from various sources, including insulin infusion devices, continuous glucose sensor devices, mobile client devices, patient owned or operated computer systems, activity tracker devices, navigation or global positioning system (GPS) devices, etc. The disclosed medical device system also obtains and processes HCP data for a population of different HCPs. In this context, the HCP data includes data that originates from, or is provided by, computer-based HCP systems, wherein the HCP data includes, without limitation: medical records, patient appointment data, patient diagnostic data, treatment plans, therapy recommendations, patient medication data, patient examination notes, etc. In accordance with certain embodiments, the aggregated patient and HCP data is processed and analyzed to generate therapy-related content that can be displayed on a website and/or via a web-based application.

An exemplary embodiment of the system described here can utilize an online (web browser based) application that serves as an HCP assistant tool. To this end, the HCP assistant can be implemented with a website that provides patient-specific therapy recommendations, advice, and/or guidance to HCPs for purposes of driving better patient outcomes. In accordance with certain implementations, the web-based tool provides physicians with valuable information (recommendations) for patients on insulin therapy, and enables physicians to make better informed decisions regarding the care of their patients, wherein at least some of those decisions are based on the analysis of patient data, medical device data, and HCP data. The HCP can also be provided with aggregated statistics related to patient behavior, glucose sensor patterns, alarm, and transmitter patterns. The web-based tool is an ideal platform to leverage machine learning algorithms that are executed by a cloud-based server system.

The web-based tool presents the end user with a variety of interactive web pages that include graphical elements, menus, text entry fields, search fields, output/results screens, and the like. For example, the web-based tool may include the following functionality, without limitation: a search page that allows the user to select or identify healthcare professionals or groups for purposes of comparing patient data; a listing or table of patients under the care of the HCP; geographical map displays that identify patient locations in connection with designated treatment and/or demographic criteria; output pages or displays that allow users to review the clinical outcomes of their own patients, with or without a comparison to the clinical outcomes of other patients; economic summary pages or displays that identify areas where the user or the user's patients can reduce healthcare costs associated with the treatment provided by the user; and recommendation pages or displays that provide tips, guidance, and suggestions to improve the treatment plan, therapy, and medical outcome of the patient. These and other features and functions enable a healthcare professional to quickly and easily review results that are based on an analysis of relevant patient data gathered for a large population of patients, wherein those results can have a positive impact on at least some of the patients under the care of that healthcare professional.

For the sake of brevity, conventional features and functionality related to infusion systems, insulin pumps, infusion sets, and fluid reservoirs may not be described in detail here. Examples of infusion pumps and/or related pump drive systems used to administer insulin and other medications may be of the type described in, but not limited to, U.S. Pat. Nos. 5,505,709; 6,485,465; 6,554,798; 6,558,351; 6,659,980; 6,752,787; 6,817,990; 6,932,584; and 7,621,893; which are herein incorporated by reference.

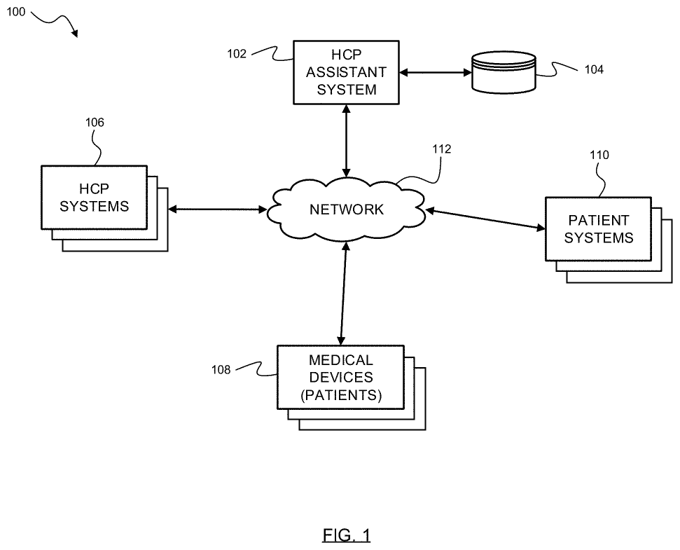

Turning now to the drawings, FIG. 1 is a simplified block diagram representation of an exemplary embodiment of a medical device system 100 that is suitably configured and arranged to support the techniques and methodologies described in more detail below. The system 100 supports HCPs and the patients under the care of the HCPs, and performs various techniques and methods to help patients manage the use of their medical devices, and to help HCPs treat and manage their patients. It should be appreciated that FIG. 1 depicts one possible implementation of a medical device system 100, and that other arrangements, architectures, and deployments can be provided if so desired. The system 100 (which has been simplified for purposes of illustration) generally includes or cooperates with the following components, without limitation: a cloud-based HCP assistant system 102 that includes or cooperates with a database system 104; one or more computer-implemented HCP user systems 106; one or more medical devices 108, which are owned or operated by one or more respective patients; and one or more computer-implemented patient systems 110, which are usually owned or operated by the patients, or by persons associated with the patients (such as parents, caregivers, etc.).

In accordance with the illustrated embodiment, the HCP assistant system 102, the HCP user systems 106, the medical devices 108, and the patient systems 110 are communicatively coupled to a network 112. This allows the HCP user systems 106, the medical devices 108, and the patient systems 110 to provide relevant data to the HCP assistant system 102. The network connectivity also enables the HCP assistant system 102 to communicate with the HCP user systems 106, the medical devices 108, and the patient systems 110 as needed. For example, certain implementations of the HCP assistant system 102 generate web pages for presentation at client devices/systems, and utilize the network 112 to communicate the web page content on demand.

FIG. 1 depicts the network 112 in a simplified manner. In practice, the system 100 may cooperate with and leverage any number of wireless and any number of wired data communication networks maintained or operated by various entities and providers. Accordingly, communication between the various components of the system 100 may involve multiple network links and different data communication protocols. In this regard, the network 112 can include or cooperate with any of the following, without limitation: a local area network; a wide area network; the Internet; a personal area network; a cellular communication network; a satellite communication network; a video services or television broadcasting network; a network onboard a vehicle; or the like. The components of the system 100 may be suitably configured to support a variety of wireless and wired data communication protocols, technologies, and techniques as needed for compatibility with the network 112.

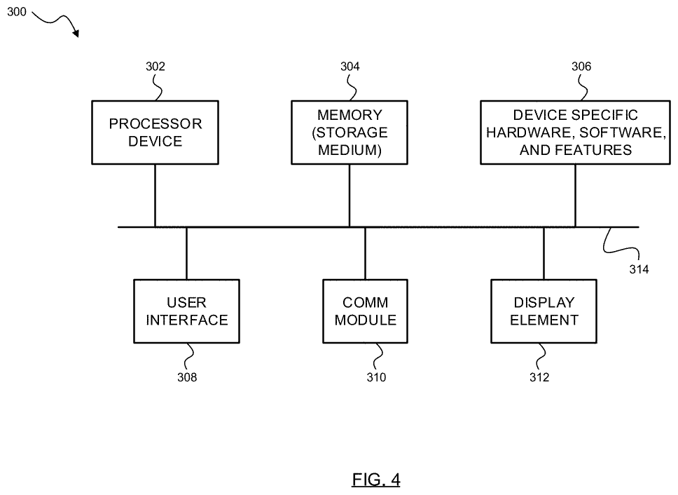

In accordance with certain exemplary embodiments, the HCP assistant system 102 and the other individual components of the system 100 are each implemented as at least one computer-based or processor-based component. For simplicity and ease of illustration, FIG. 1 depicts the HCP assistant system 102 as a single block—it should be appreciated that any number of distinct hardware components can be utilized to implement the HCP assistant system 102. An exemplary embodiment of a device suitable for implementing the HCP assistant system 102 is described below with reference to FIG. 4.

Each medical device 108 is a hardware component that is designed, configured, and operated to provide some form of therapy to a patient. Although this disclosure contemplates different types and form factors of medical devices 108, the exemplary embodiment presented here relates to a diabetes management system where the medical devices 108 are insulin infusion devices, glucose sensor devices, glucose meters, glucose monitors, or other hardware components that are typically utilized in connection with the treatment of diabetes.

The HCP assistant system 102 can be considered the “heart” of the medical device system 100. The HCP assistant system 102 includes or cooperates with the database system 104 (which is realized using one or more components), which is configured to support the functionality and operation of the HCP assistant system 102. The HCP assistant system 102 collects and analyzes input data for each HCP and for each patient (the input data can originate from various sources, including an insulin infusion device and/or a source other than the insulin infusion device, such as: a glucose sensor or meter, a mobile device operated by a user of the insulin infusion device, a computing device, etc.), generates relevant and timely messages and recommendations as needed, and manages the delivery of the generated messages to the HCPs and patients. The HCP assistant system 102 and the related database system 104 are described in more detail below.

In certain embodiments, some or all of the functionality and processing intelligence of the HCP assistant system 102 can reside at the HCP user systems 106. In other words, the medical device system 100 need not rely on a network-based or a cloud-based server arrangement, although such a deployment might be the most efficient and economical implementation. This and other alternative arrangements are contemplated by this disclosure. To this end, some embodiments of the system 100 may include additional devices and components that serve as data sources, data processing units, and/or message delivery mechanisms.

Each HCP user system 106 and each patient system 110 can be realized using a variety of different device platforms. For example, these systems can be implemented as any of the following, without limitation: a cellular telephone or smartphone; a portable computer (e.g., a laptop, a tablet, or a netbook computer); a portable media player; a portable video game device; a portable medical device; a navigation device such as a global positioning system (GPS) device; a wearable computing device; an electronic toy or game; a smart home appliance; a smart television set; a vehicle-based computing module; etc. In accordance with certain exemplary embodiments, each HCP user system 106 and each patient system 110 supported by the medical device system 100 is implemented as a computer-based or processor-based component. An exemplary embodiment of a device suitable for implementing an HCP user system 106 or a patient system 110 is described below with reference to FIG. 4.

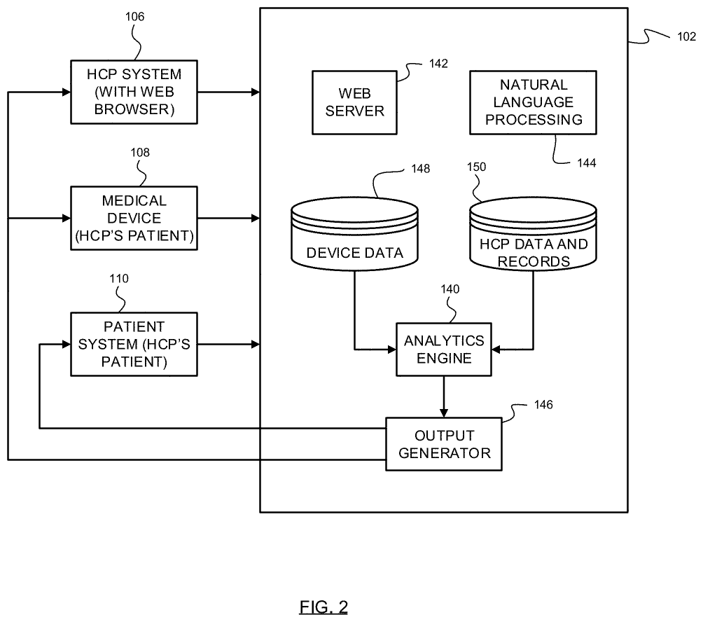

The HCP assistant system 102 is scalable to support a plurality of different HCPs and a plurality of different HCP user systems 106. In a practical deployment, a physician (i.e., an HCP user of the system 100) owns or operates at least one HCP user system 106, which may be a portable computer or a desktop computer located at the physician's office. In this regard, FIG. 2 is a block diagram that depicts the HCP assistant system 102 in more detail, and in association with only one HCP user system 106, only one medical device 108, and only one patient system 110. The data communication network 112 is not shown in FIG. 2. For this particular scenario, the HCP who owns or operates the HCP user system 106 is considered to be the target end user of the HCP assistant system 102. Although the HCP may have a number of different patients, the following description of FIG. 2 focuses on only one patient—the patient who uses the medical device 108 and the patient system 110.

As shown in FIG. 2, the exemplary embodiment of the HCP assistant system 102 includes or cooperates with: an analytics engine 140; a web server 142; a natural language processing module 144; an output generator 146; a database system 148 to receive, collect, and maintain medical device user data; and a database system 150 to receive, collect, and maintain HCP data and records. The components, elements, and functional modules of the HCP assistant system 102 are suitably arranged and configured to communicate and cooperate with one another as needed to support the various features and functionality described in more detail herein.

The analytics engine 140 accesses and analyzes medical device user data stored in the database system 148 and HCP data and records stored in the database system 150. The analytics engine 140 processes and analyzes the device user data, HCP data, and medical records data using, for example: machine learning algorithm(s); expert system technology; artificial intelligence techniques; a knowledge base; natural language processing; and/or similar methodologies. The results from the analytics engine 140 are provided to the output generator 146, which generates messages, alerts, recommendations, notifications, instructions, reminders, and other content for distribution to the HCP user system 106, the medical device 108 and/or the patient system 110 as needed. The output generator 146 may cooperate with the web server 142 to convey the generated output in the form of web pages to be displayed at a client-side device. The output generator 146 may cooperate with the natural language processing module 144 to create and format appropriate text or audio content to be delivered to a client-side device.

The output generated by the HCP assistant system 102 enables HCPs to monitor patient progress in meeting certain behavioral and/or clinical outcome goals. Although the output generated by the exemplary embodiment contemplated here is conveyed in the form of an HTML document (a web page), the output generator 146 may alternatively or additionally generate output that is configured, arranged, and formatted for delivery by alternative mechanisms, such as email, text messaging, direct notifications to a client device or an application running on a client device, a voice message, or the like. The HCP assistant system 102 can generate useful output (graphs, charts, reports, notifications, statistics related to patient behavior, statistics related to medical device usage or performance, statistics related to medical device alarms or alerts, and so on) that are intended to benefit the HCPs and the patients. In certain implementations, the HCP assistant system 102 utilizes the data analytics and insight delivery system and methodologies disclosed in United States Patent Application Publication number US 2017/0053072, the content of which is incorporated by reference herein.

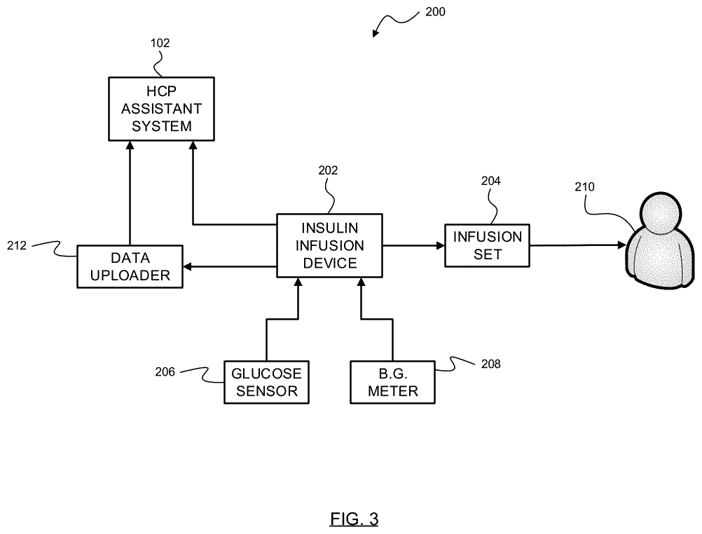

FIG. 3 is a block diagram of a medical device system 200 that includes an insulin infusion device 202 and associated components. The system 200 corresponds to an implementation of the medical device system 100, where the patient is a user of an insulin infusion system that includes the insulin infusion device 202 and related devices/systems. In this regard, the system 200 depicted in FIG. 3 is consistent and compatible with the alternative representations depicted in FIG. 1 and FIG. 2; the medical devices 108 shown in FIG. 1 and FIG. 2 include the components of the insulin infusion system shown in FIG. 3. Accordingly, FIG. 3 includes the HCP assistant system 102 described previously. As mentioned above, the HCP assistant system 102 can support many different users of insulin infusion devices, and performs various techniques and methods to manage and control the use of insulin infusion devices. For simplicity, however, FIG. 3 only depicts the insulin infusion system for one particular patient who is under the care of one target user of the HCP assistant system 102 (one specific caregiver, physician, nurse, or the like).

It should be appreciated that FIG. 3 depicts one possible implementation of the system 200, and that other arrangements, architectures, and deployments can be provided if so desired. The system 200 (which has been simplified for purposes of illustration) generally includes or cooperates with the following components, without limitation: the HCP assistant system 102; the insulin infusion device 202; an infusion set 204; a continuous glucose sensor 206; and a blood glucose (BG) meter 208. The insulin infusion device 202, the infusion set, the glucose sensor 206, and the BG meter 208 are components of the insulin infusion system that is used by patient 210 to treat diabetes. In certain implementations, the system 200 may also include or cooperate with an optional data uploader component 212.

At least some of the components of the system 200 are communicatively coupled with one another to support data communication as needed. For this particular example, the HCP assistant system 102 and the insulin infusion device 202 communicate with each other via a suitable data communication network (which is not depicted in FIG. 3). Moreover, the data uploader component 212 is preferably configured as an interface component that communicates data from the insulin infusion device 202 to the HCP assistant system 102 using a suitable data communication network. In certain embodiments, the insulin infusion device 202 and/or the continuous glucose sensor 206 are communicatively coupled to the network to facilitate the uploading of relevant data directly to the remote HCP assistant system 102. Alternatively, or additionally, the insulin infusion device 202 provides relevant data directly to the data uploader component 212, which in turn uploads the data to the HCP assistant system 102 via the network. Other configurations and topologies are also contemplated here, such as a system that includes one or more intermediary, interface, or data repeating devices in the data path between the HCP assistant system 102 and the insulin infusion device 202.

FIG. 3 depicts network communication links in a simplified manner. In practice, the system 200 may cooperate with and leverage any number of wireless and any number of wired data communication networks maintained or operated by various entities and providers. Accordingly, communication between the various components of the system 200 may involve multiple network links and different data communication protocols. In this regard, the network can include or cooperate with any of the following, without limitation: a local area network; a wide area network; the Internet; a personal area network; a cellular communication network; a satellite communication network; a video services or television broadcasting network; a network onboard a vehicle; or the like. The components of the system 200 may be suitably configured to support a variety of wireless and wired data communication protocols, technologies, and techniques as needed for compatibility with the network.

In certain embodiments, the insulin infusion device 202 is a portable patient-worn or patient-carried component that is operated to deliver insulin into the body of the patient 210 via, for example, the infusion set 204. In accordance with certain exemplary embodiments, each insulin infusion device 202 supported by the system 200 is implemented as a computer-based or processor-based component. For simplicity and ease of illustration, FIG. 3 depicts only one insulin infusion device 202. In practice, however, the system 200 is suitably configured to support a plurality of insulin infusion devices 202, wherein each patient or user owns or operates at least one of the insulin infusion devices 202. An exemplary embodiment of a device suitable for implementing the insulin infusion device 202 is described below with reference to FIG. 4.

The HCP assistant system 102 obtains input data from one or more sources, which may include various diabetes management devices (the insulin infusion device 202, a continuous glucose monitoring device, the glucose sensor 206, a monitor device, or the like). In this regard, the insulin infusion device 202 represents a source of input data for the HCP assistant system 102. In certain embodiments, the insulin infusion device 202 provides data that is associated with its operation, status, insulin delivery events, and the like. As mentioned previously, the relevant data generated or collected by the insulin infusion device 202 can be transmitted directly to the HCP assistant system 102 or indirectly by way of the data uploader component 212, depending on the particular implementation of the system 200. The particular type of data provided by the insulin infusion device 202 is described in more detail below.

For the sake of simplicity, FIG. 3 depicts only one glucose sensor 206. In practice, however, the system 200 is suitably configured to support a plurality of glucose sensors 206, wherein each patient or user owns or operates at least one of the glucose sensors 206. The glucose sensor 206 is suitably configured to measure a glucose level (interstitial) of the patient in real time. The glucose sensor 206 may include a wireless transmitter that facilitates transmission of the sensor glucose data to other devices, such as the insulin infusion device 202 or the data uploader component 212. In some implementations, the glucose sensor 206 can provide the sensor glucose data directly to the HCP assistant system 102 if so desired.

For the sake of simplicity, FIG. 3 depicts only one BG meter 208. In practice, however, the system 200 is suitably configured to support a plurality of BG meters 208, wherein each patient or user owns or operates at least one of the BG meters 208. The BG meter 208 is configured to measure the blood glucose level of a user by analyzing a blood sample. For example, the BG meter 208 may include a receptacle for receiving a blood sample test strip. In this regard, the user inserts a test strip into the BG meter 208, which analyzes the sample and displays a blood glucose level corresponding to the test strip sample. The BG meter 208 may be configured to communicate the measured blood glucose level to the insulin infusion device 202 for storage and processing, directly to the HCP assistant system 102, or to the data uploader component 212. In some scenarios, the patient is responsible for entering each blood glucose measurement into the insulin infusion device 202. Ultimately, the measured blood glucose data is provided to the HCP assistant system 102 for analysis.

Depending on the particular embodiment and application, the medical device system 200 can include or cooperate with other devices, systems, and sources of input data. For example, in certain embodiments the system 200 includes one or more sources of contextual information or data, which may include, without limitation: activity tracker devices; meal logging devices or apps; mood tracking devices or apps; and the like.

Referring again to FIG. 2, the HCP assistant system 102 obtains data from various sources such that the analytics engine 140 can review and process the collected data in an appropriate manner to generate the desired type of output for distribution to HCPs and patients. For ease of description and convenience, FIG. 2 depicts a database system 148 for device data and a database system 150 for HCP data and records. In practice, the HCP assistant system 102 may include a single database to accommodate all types of data, or it may include one or more databases that are arranged and maintained as needed to accommodate the different types of data.

Data Types and Sources

The HCP assistant system 102 is suitably configured to receive and process a variety of input data from multiple sources. Moreover, the system 102 is designed to be flexible and scalable to accommodate additional input data types as needed. The number of input data sources and the amount of input data handled by the system 102 may vary from one embodiment to another, depending on the particular implementation and intended application. The medical device user data received by the HCP assistant system 102 (and maintained in the database system 148) includes data generated by, provided by, or originating at devices or systems associated with the patients. These patient devices and systems may include, without limitation, medical devices owned or used by the patients, smartphones, personal computers, activity tracker devices, GPS devices, vehicle-originated data, smart home systems or components, smart appliances, and the like. In contrast, the HCP data and records received by the HCP assistant system 102 (and maintained in the database system 150) includes data generated by, provided by, or originating at devices or systems associated with the various HCPs, including specific HCP customers. These HCP devices and systems may include, without limitation, medical equipment or medical devices owned or operated by the HCPs, smartphones, personal computers, smart office systems or components, smart appliances, and the like.

The device data (for the exemplary deployment with an insulin infusion system) includes data related to BG meter measurements, glucose sensor readings, insulin delivery information, and other information that is associated with the use of the insulin infusion device. Although the HCP assistant system 102 obtains and analyzes such data, it may also obtain and consider additional data, including information collected and provided by one or more mobile applications resident on the patient's mobile device. The HCP assistant system 102 may also process data received directly or indirectly from other physiological sensors, devices, or equipment. For example, an embodiment of the HCP assistant system 102 can be suitably configured to analyze respiratory data, electrocardiogram data, body temperature data, heartrate information, and the like.

In accordance with certain embodiments, some or all of the following device data can be used as input to the analytics engine 140 of the HCP assistant system 102 for purposes of generating insight messages, prioritizing the delivery of generated messages, generating glucose management recommendations, and the like. The following summary of specific types of device data is not intended to be exhaustive or otherwise limiting, and alternative or additional device data can be considered in an embodiment of the HCP assistant system 102.

Carbohydrate amount—this refers to the carbohydrate amount that one Unit of insulin can compensate to maintain the current glucose level. The carbohydrate amount is usually expressed in grams or milligrams. The patient's mobile device or insulin infusion device will usually be the source of this data.

Bolus information—the bolus information includes the bolus dosage amount (in Units of insulin), the date/time of delivery (time of day and calendar data), and the bolus type (normal, square, or dual). The insulin infusion device will usually be the source of this data.

Insulin to carbohydrate ratio—this is a patient-specific parameter that relates to how much insulin the patient needs to compensate for a designated unit (e.g., one gram) of carbohydrate. The insulin to carbohydrate ratio is expressed in grams/Unit. The insulin infusion device will usually be the source of this data.

Insulin sensitivity factor—this is a patient-specific parameter that relates to the reduction in blood glucose in response to one Unit of insulin. The particular manner in which the insulin sensitivity factor is calculated is determined by the specific pumping protocol. The insulin sensitivity factor is expressed in mg/dL/U (milligrams per deciliter per Unit). The insulin infusion device will usually be the source of this data.

Active insulin amount—this refers to how much insulin is still active in the body of the patient from previous bolus doses. This quantity is expressed in Units of insulin. The insulin infusion device will usually be the source of this data.

Time of day—this refers to timestamp and/or date stamp information, which may be associated with or appended to any other piece of input data to provide a time reference.

Basal rate—this is a patient-specific parameter that indicates the basal rate of insulin delivery, which is usually expressed in Units/hour. The insulin infusion device will usually be the source of this data.

Temporary basal use—this refers to an occurrence during which the patient temporarily “overrides” the nominal or usual basal rate of insulin. The insulin infusion system employs a boolean value that indicates the activation of the temporary basal mode, and also indicates the temporary basal rate value. The insulin infusion device will usually be the source of this data.

Consecutive boluses—this refers to an occurrence of back-to-back insulin boluses, which are delivered within a designated period of time. The insulin infusion system employs a boolean value that indicates the occurrence of consecutive boluses, and also indicates the total volume of the boluses delivered during the designated period of time. The insulin infusion device will usually be the source of this data.

Insulin suspension—this refers to a period of time during which the insulin infusion device has been temporarily suspended (insulin delivery is temporarily halted). The data related to insulin suspension can include some or all of the following, without limitation: threshold setting; suspension duration; active insulin before the suspension; sensor rate of change around the suspension; carbohydrate intake around the suspension; time (day of week, time of day) of the suspension; how the suspension recovered; and user response to the suspension. The insulin infusion device will usually be the source of this data.

Reservoir rewind and priming time—this refers to activities associated with the installation of a new insulin reservoir into the insulin infusion device. This requires a rewind action to retract the reservoir actuator, which facilitates removal of the used reservoir. After installing the new reservoir, the fluid flow path is primed for insulin delivery. The insulin infusion device will usually be the source of this data.

Pump alarms and associated alarm times—pump alarms can be generated by the insulin infusion device for various reasons. Pump alarm data indicates the type of alarm and the corresponding alarm time. The insulin infusion device will usually be the source of this data.

Sensor alerts and alert times—sensor alerts can be generated by the insulin infusion device and/or the glucose sensor for various reasons. Sensor alert data indicates the type of sensor alert and the corresponding alert time. The insulin infusion device and/or the glucose sensor can be the source of this data.

Blood glucose readings and measurement times—blood glucose readings are usually expressed in mg/dl, and are obtained from a blood glucose meter. The insulin infusion device, the blood glucose meter, or the patient's mobile device can be the source of this data.

User demographic information—this data may include, without limitation, the patient's age, number of years using insulin, medical diagnosis, age at the onset of diabetes, sex, medication types, and the like. User demographic information can be provided by the patient's mobile device, the insulin infusion device, a webpage user interface, or the like.

Meal time and content—this data relates to the timing of meal consumption and the type and amount of food consumed. The patient's mobile device will usually be the source of this data. In this regard, a suitably configured mobile app can include a feature or functionality that allows the patient to specify meal times and to estimate the type and amount of food consumed at each meal. In certain scenarios, this data can be imported from a third party (partner) database directly, rather than having patients redundantly enter the information into the mobile app.

Exercise time and content—this data relates to the timing of exercise and the type, duration, and amount of exercise performed by the patient. The patient's mobile device will usually be the source of this data. In this regard, a suitably configured mobile app can include a feature or functionality that allows the patient to specify exercise times and to estimate the type and amount of exercise. In certain scenarios, this data can be imported from a third party (partner) database directly, rather than having patients redundantly enter the information into the mobile app.

Medication type, dosage, and time—this data relates to instances when the patient takes medication (other than insulin), and the data indicates the type of medication, the dosage taken, and the time that the medication was taken. The patient's mobile device will usually be the source of this data. In some scenarios, a smart insulin pen or other type of smart insulin delivery device can be the source of this data. In this regard, a suitably configured mobile app can include a feature or functionality that allows the patient to record information associated with taking medication.

Sleep time and quality—this data indicates sleeping periods, and information related to the quality or type of sleep experienced by the patient. The sleep-related information can be provided by a patient monitor or, in certain embodiments, the sleep-related information is provided by a suitably configured mobile app running on the patient's mobile device. In such an embodiment, the mobile app allows the patient to enter the relevant sleep-related information. In accordance with some embodiments, sleep related information can be calculated using accelerometer data, heartrate data, ambient lighting measurements, glucose levels, etc.

Stress time—this data indicates periods of stress experienced by the patient. The stress-related information can be derived from physiological factors and/or measurable data such as heart rate, blood pressure, skin conductance, body temperature, or the like. Additionally or alternatively, the stress-related information can be based on user input. Accordingly, the patient's mobile device can be the source of this data. A suitably configured mobile app can include a feature or functionality that allows the patient to record information associated with periods of stress.

User reaction to delivered insight messages and content—this data represents user feedback, and it may be considered to be a form of input data. The patient's mobile device will usually be the source of this data. In this regard, a suitably configured mobile app can include a feature or functionality that allows the patient to provide user feedback related to glycemic insights delivered to the patient.

User behavior change to delivered insight messages and content—this data is associated with actions taken by the patent in response to insight messages delivered by the system. User behavior change is measured by the percent of change of the occurrence of those events that were communicated to users as glycemic insights. It indicates whether the insight messages provide any impact on user's behavior and whether the behavior change leads to significant outcome improvement.

Mobile device data—the patient's mobile device or a mobile app can manage and upload the following information, without limitation: calendar data (time of day, day of the week, month, season, etc.); user profile data; GPS data that indicates the geographic position of the mobile device; map or navigation data associated with operation of the mobile device; user-entered meal consumption, food content, and/or food ingredient data; user-entered carbohydrate data; user-entered exercise related data; user-entered medication related data; user response data associated with the receipt of glycemic insight messages; user feedback related to glycemic insight messages; accelerometer data; contacts list information; web browser data; consumer purchasing data; and the like.

An HCP can enter medical data that is associated with patients. The HCP user system 106 may provide a web browser interface that includes web pages configured to accept data entered or uploaded by the HCP. In certain embodiments, the HCP data is limited and structured in that only certain types or categories of HCP data can be input. In other embodiments, the natural language processing module 144 can be suitably configured to receive and process “free form” data entered by the HCP. In accordance with certain embodiments, some or all of the following HCP data and records can be used as input to the analytics engine 140 of the HCP assistant system 102 for purposes of generating insight messages, prioritizing the delivery of generated messages, generating glucose management recommendations, and the like. The following summary of specific types of HCP data and records is not intended to be exhaustive or otherwise limiting, and alternative or additional data can be considered in an embodiment of the HCP assistant system 102.

Patient medication data and prescription logs—this data relates to medications used by the HCP's patients, including dosages, prescriptions, schedules, etc.

Electronic patient medical records and medical lab test data—this data can be provided by the HCP system, medical facilities, insurance companies, or the like.

Mobile device data—the HCP's mobile device or a mobile app can manage and upload the following information, without limitation: calendar data (time of day, day of the week, month, season, etc.); user profile data; GPS data that indicates the geographic position of the mobile device; map or navigation data associated with operation of the mobile device; contacts list information; web browser data; and the like.

Patient appointment data—this data relates to patient appointments, and it may include, without limitation: calendar entries; appointment scheduling information; agenda items or topics to be discussed during patient appointments; and the like.

Therapy-related notes, recommendations, and observations—this data may include, without limitation: caregiver or physician treatment notes; patient diagnostic data and related notes; treatment plans; therapy recommendations; observed patient symptoms; patient examination notes; and the like.

Patient goals and objectives—this data relates to the desired medical or therapy outcomes of the patients.

Miscellaneous Metrics and Indicators—this data relates to other patient-related or background information, including, without limitation: age; gender; diabetes indication (Type 1 or Type 2); time since first diagnosis; body weight; body mass index; height; blood pressure records; comorbidities; other medical diagnoses; emergency room records; etc.

As mentioned above, the system 100 includes or cooperates with computer-based, computer-implemented, and/or processor-based components having suitably configured hardware and software written to perform the functions and methods needed to support the features described herein. For example, the HCP assistant system 102, each HCP user system 106, each patient system 110, and any of the medical devices 108 can be realized as an electronic processor-based component. In this regard, FIG. 4 is a simplified block diagram representation of an exemplary embodiment of a computer-based or processor-based device 300 that is suitable for deployment in the system shown in FIG. 1.

The illustrated embodiment of the device 300 is intended to be a high-level and generic representation of one suitable platform. In this regard, any of the computer-based or processor-based components of the system 100 can utilize the architecture of the device 300. The illustrated embodiment of the device 300 generally includes, without limitation: at least one processor device 302; a suitable amount of memory 304; device-specific hardware, software, firmware, and/or features 306; a user interface 308; a communication module 310; and a display element 312. Of course, an implementation of the device 300 may include additional elements, components, modules, and functionality configured to support various features that are unrelated to the subject matter described here. For example, the device 300 may include certain features and elements to support conventional functions that might be related to the particular implementation and deployment of the device 300. In practice, the elements of the device 300 may be coupled together via a bus or any suitable interconnection architecture 314.

The processor device 302 may be implemented or performed with a general purpose processor, a content addressable memory, a digital signal processor, an application specific integrated circuit, a field programmable gate array, any suitable programmable logic device, discrete gate or transistor logic, discrete hardware components, or any combination designed to perform the functions described here. Moreover, the processor device 302 may be implemented as a combination of computing devices, e.g., a combination of a digital signal processor and a microprocessor, a plurality of microprocessors, one or more microprocessors in conjunction with a digital signal processor core, or any other such configuration.

The memory 304 may be realized as RAM memory, flash memory, EPROM memory, EEPROM memory, registers, a hard disk, a removable disk, a CD-ROM, or any other form of storage medium known in the art. In this regard, the memory 304 can be coupled to the processor device 302 such that the processor device 302 can read information from, and write information to, the memory 304. In the alternative, the memory 304 may be integral to the processor device 302. As an example, the processor device 302 and the memory 304 may reside in an ASIC. At least a portion of the memory 304 can be realized as a computer storage medium that is operatively associated with the processor device 302, e.g., a tangible computer-readable medium having computer-executable instructions stored thereon. The computer-executable instructions, when read and executed by the processor device 302, cause the device 300 to perform certain tasks, operations, functions, and processes that are specific to the particular embodiment. In this regard, the memory 304 may represent one suitable implementation of such computer-readable media. Alternatively or additionally, the device 300 could receive and cooperate with computer-readable media (not separately shown) that is realized as a portable or mobile component or platform, e.g., a portable hard drive, a USB flash drive, an optical disc, or the like.

The device-specific hardware, software, firmware, and features 306 may vary from one embodiment of the device 300 to another. For example, the device-specific hardware, software, firmware, and features 306 will support: insulin pump operations when the device 300 is realized as an insulin infusion device; server system operations when the device 300 is realized as a cloud-based computing device; etc. In practice, certain portions or aspects of the device-specific hardware, software, firmware, and features 306 may be implemented in one or more of the other blocks depicted in FIG. 4.

The user interface 308 may include or cooperate with various features to allow a user to interact with the device 300. Accordingly, the user interface 308 may include various human-to-machine interfaces, e.g., a keypad, keys, a keyboard, buttons, switches, knobs, a touchpad, a joystick, a pointing device, a virtual writing tablet, a touch screen, a microphone, or any device, component, or function that enables the user to select options, input information, or otherwise control the operation of the device 300. The user interface 308 may include one or more graphical user interface (GUI) control elements that enable a user to manipulate or otherwise interact with an application via the display element 312.

The communication module 310 facilitates data communication between the device 300 and other components as needed during the operation of the device 300. In the context of this description, the communication module 310 can be employed to transmit or stream device-related control data, patient-related data, device-related status or operational data, therapy recommendations, infusion device adjustment recommendations and related control instructions, and the like. It should be appreciated that the particular configuration and functionality of the communication module 310 can vary depending on the hardware platform and specific implementation of the device 300. In practice, an embodiment of the device 300 may support wireless data communication and/or wired data communication, using various data communication protocols. For example, the communication module 310 could support one or more wireless data communication protocols, techniques, or methodologies, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. Moreover, the communication module 310 could support one or more wired/cabled data communication protocols, including, without limitation: Ethernet; powerline; home network communication protocols; USB; IEEE 1394 (Firewire); hospital network communication protocols; and proprietary data communication protocols.

The display element 312 is suitably configured to enable the device 300 to render and display various screens, recommendation messages, notifications, GUIs, GUI control elements, drop down menus, auto-fill fields, text entry fields, message fields, or the like. Of course, the display element 312 may also be utilized for the display of other information during the operation of the device 300, as is well understood. Notably, the specific configuration, operating characteristics, size, resolution, and functionality of the display element 312 can vary depending upon the practical implementation of the device 300.

The general architecture depicted in FIG. 4 can be utilized by the insulin infusion device 202. A suitably configured, designed, and programmed insulin delivery controller of the insulin infusion device 202 can be realized using the processor device 302, the memory 304, and/or the device specific hardware, software, and features 306. Alternatively, the insulin delivery controller can be realized as a microcontroller device, an application-specific integrated circuit (ASIC), or the like.

The medical device system 100 can be utilized as a cloud-based hub that delivers therapy-related insight messages to patients, in a manner that is controlled by, influenced by, or regulated by the HCPs who are responsible for the care of the patients. In other words, a patient's HCP can determine whether or not certain insight messages are delivered to the patient and, if so, at what time. In this regard, the system collects all insight messages (delivered messages or messages that have been generated but not yet delivered) for patients of the target HCP user, and displays the messages in both an aggregated and individualized view on, for example, a web page that can be accessed by the HCP. Insight messages can be visualized and displayed in accordance with the HCP's settings or preferences, such as aggregated by patients, aggregated by insight type, arranged by timeline, or arranged by severity or urgency. An insight message curation methodology can be added to the system to display high priority insight messages to the HCP in accordance with certain settings and preferences. The target HCP user may be provided with different settings to approve the delivery of insight messages to patients. These settings may include, without limitation: deliver all insight messages automatically; only deliver insight messages in certain categories, such as non-therapy adjustment related insight messages; deliver insight messages only if they are manually approved by the HCP. The outcomes of delivered insight messages can be monitored and fed back to the system so that results can be displayed or otherwise presented to the HCP.

The medical device system 100 reduces the regulatory burden of delivering insight messages to patients. The system also creates a bridge between physicians and the intelligent insight message system, which improves trust between the two parties. The system alleviates the HCP's burden of observing and creating therapy adjustment suggestions, because much of the underlying analysis and decision-making can be automated. Moreover, the medical device system collects and reviews data associated with HCP behavior and tendencies related to certain therapy adjustments, which can be used to improve the system in an ongoing manner.

As mentioned above, embodiments of the described medical device system can employ an intelligent web or mobile-based program that allows HCPs to conduct their appointments more efficiently. In addition to having standard electronic medical record functionalities, such as viewing upcoming appointments and managing appointment reminders, the HCP assistant system helps guide the physician through the appointment itself, helping the HCP prioritize the most important clinical aspects for a specific patient. The backend infrastructure of the HCP assistant system reviews the patient's continuous glucose monitoring data, in addition to the patient's contextual data (e.g., nutrition, fitness and medication logs), to determine which topics will be the most relevant to cover during the appointment. For example, one patient may need to focus more on insulin titration, while another may need to focus more on diet and exercise. At the end of the visit, the HCP assistant system provides the physician with recommendations on next steps, and delivers any relevant educational materials to the patient. In certain embodiments, the HCP assistant system includes a voice or text enabled chat functionality that is suitably designed to quickly find and display any relevant information that the physician may need to view during a patient visit. The HCP assistant system is intelligent enough to remind patients of any follow-up actions they need to take between visits, and is also able to automatically alert the physician and his or her staff if the patient is experiencing any medical conditions that may require intervention (e.g., hypoglycemia or ketoacidosis). These and other features save the physician time both during and between patient visits, allowing the physician to spend less time on clerical tasks and more time on delivering better quality care.

Appointment Navigator

The appointment navigator feature of the HCP assistant system 102 relies on machine learning and artificial intelligence to create a personalized “journey” for each physician and his or her patient during an appointment. The appointment navigator feature prioritizes the most relevant topics for that specific patient during that specific appointment, wherein the prioritization scheme is based on the input data accessed by the analytics engine 140. The appointment navigator feature can also perform any of the following, without limitation: schedule appointments with patients; access and present relevant patient reports, lab results, and/or medical records that may be useful during a patient visit; obtain and present relevant research articles, medical papers, medical journal submissions, and/or other documents that may be useful during a patient visit; and access insight messages that have been delivered (or are scheduled to be delivered) to the patient, and present those messages to the physician so that they can be discussed with the patient during an appointment.

The appointment navigator feature considers preferences, historical appointment information, and behavior of each HCP user to help manage appointment scheduling, appointment agendas, and other aspects of patient appointments. For example, the appointment navigator feature can recommend when the next appointment for a given patient should be, based on the HCP user's tendencies and past behavior. Appointments can be managed more efficiently based on historical patient data, HCP preferences, the current patient condition (which may be deduced from collected patient or device data), and other factors. The appointment navigator feature can automatically determine how long a scheduled patient appointment should be, and the length of time between consecutive appointments.

Prescription and Supplies Reminder

The system may also implement a reminder feature for patient prescriptions, medical supplies, and/or other consumables. To this end, the reminder feature can generate a reminder message or notification (intended for the patient or the HCP) when the patient is running low on supplies, if a prescription needs to be refilled, if a prescription is about to expire, or the like. The reminder feature can also alert the HCP if a new version of a prescribed device is available for use, and recommend that the patient upgrade to the new version. Similarly, the reminder feature can notify the HCP if a generic version of a prescription drug is available and/or if alternatives or equivalents to a currently prescribed medication are available for consideration.

Clinical Watch

The clinical watch feature keeps an automatic watch over the physician's patients in an ongoing manner. In certain implementations, the clinical watch feature is realized as one or more web pages that include a list of the HCP's patients, wherein further details of each patient can be accessed or viewed on demand. The clinical watch feature checks whether any patients experience a notable medical event between physician visits. In addition, the clinical watch feature automatically communicates with patients as needed to check whether any assistance is needed. The clinical watch feature can generate notifications, alerts, and patient status updates to the HCP user systems 106 as needed or on demand. For example, if a patient needs medical care or attention, an appropriate notification or message can be automatically sent to the patient's HCP for viewing at the respective HCP user system 106.

Chatbot

The chatbot feature allows a physician to quickly pull any relevant patient information they may need, both during and between patient visits, simply by speaking into their device's microphone or typing a request on the frontend of the HCP assistant system 102 (e.g., text entry fields that appear on a web page). The HCP assistant system 102 is able to parse the physician's voice or text command, retrieve the requested information, and display it on the device's display. The chatbot feature can leverage machine learning, artificial intelligence, natural language processing, and associated technologies to facilitate two-way discussions between the chatbot feature and patients, two-way discussions between the chatbot feature and HCPs, and three-way discussions between the chatbot feature, patients, and HCPs. Moreover, for pediatric patients, the chatbot feature can involve parents or other third party chat participants. On the HCP side, the chatbot feature can involve nurses, physician assistants, staff members, customer support personnel, or the like. As one non-limiting example, an HCP can utilize the chatbot feature as a way of directing and/or utilizing the appointment navigator feature mentioned above.

Medical Device Commands

In accordance with certain optional embodiments, the HCP assistant system 102 generates suitably formatted control instructions, commands, configuration settings, and the like, which are intended for the patient's medical device. In such embodiments, the HCP user system can be operated by the HCP user to initiate automatic adjustments of the patient's medical device in accordance with certain therapy adjustment recommendations generated by the HCP assistant system 102. Thus, the HCP assistant system 102 could be deployed and utilized to change therapy-related settings of the medical device, initiate or remotely control the delivery of therapy by the medical device (e.g., deliver medication fluid to the patient), generate alerts or alarms at the medical device, send messages to be displayed or presented by the medical device, etc. Such actions can be performed automatically by the HCP assistant system 102 in response to activity of the analytics engine 140, or they can be performed after approval by the physician.

Analytics Engine

The analytics engine 140 learns and adapts in response to device data, HCP data, and medical records data uploaded by patients and physicians. This enables the HCP assistant system 102 to provide useful recommendations to help the physician make faster and better decisions. In certain implementations, the analytics engine 140 is an adaptive learning element that understands and learns the behavior of the HCP user, such that the HCP assistant system 102 can accurately and reliably anticipate how the HCP user will react and respond to certain patient scenarios. This allows the HCP assistant system 102 to present information to the HCP user, receive feedback related to how the HCP user actually responded to the information, and dynamically adapt the analytics engine 140 in accordance with the obtained feedback. The inputs to the analytics engine 140 were summarized above. The output of the analytics engine 140 include, without limitation: predicted physician behavior related to treatment of patients; recommendations intended for the physician; information related to what other physicians have done under similar circumstances, and the associated patient outcomes.

Insight Message Manager

For the exemplary embodiment described here, the HCP user system 106 includes a web browser or a devoted program or application that receives, manages, and displays insight messages that are intended for patients of the HCP user. The HCP assistant system 102 generates patient insight messages for the HCP user's patients, and at least some of the generated insight messages are viewable by way of the HCP user system 106. This example assumes that all insight messages are viewable on the HCP user system 106, such that the HCP user can review the messages, make comments or notes regarding the messages, approve or otherwise regulate delivery of the messages to the patients, etc.

In certain embodiments, the insight message manager feature allows the HCP user to configure settings that determine how patient insight messages are handled. For example, the HCP user can configure the insight message manager such that certain types of insight messages are automatically delivered to the HCP user's patients (without requiring approval or any other action by the HCP user). As another example, the HCP user can configure the insight message manager such that certain types of insight messages must be reviewed and approved before they can be delivered to patients. In that scenario, the HCP assistant system is operated such that it delivers the patient insight message to the patient only after receiving a delivery confirmation message, instruction, or command from the HCP user system, wherein the delivery confirmation is generated or otherwise initiated by the HCP user after approving the insight message. The insight message manager feature may also include various delivery rules that can be configured by the HCP user for purposes of regulating how patient insight messages are handled, when they are delivered to the patients, and the like. For example, if a patient insight message recommends changing the insulin sensitivity setting of the patient's insulin infusion device by less than 10%, then that message can be delivered to the patient without review by the HCP user; if the recommended change is greater than 10%, then the HCP user must review the insight message and approve it before it can be delivered. As another example, the HCP user may configure the insight message manager such that routine patient insight messages that do not recommend or require any changes to the patient's therapy (such as appointment reminders, glucose level notifications, or patient status reports) are automatically delivered as soon as they are generated. In this manner, the HCP assistant system can be operated to deliver patient insight messages to patients in accordance with the delivery rules that are configured or designated by the respective HCP user systems.

Exemplary Use Cases

A typical use case will be described with reference to the general operating environments shown in FIG. 1 and FIG. 2. For this example, one particular HCP (the “HCP user”) is considered to be the target user of the system 100, and one particular patient (the “HCP patient”) is under the care of the HCP user. The system 100 also supports a population of additional HCPs and a population of additional patients falling under the care of those HCPs. In accordance with this example, the HCP user enters or uploads medical data, patient information, and related data to the HCP assistant system 102, using the respective HCP user system 106. The HCP patient also uploads device data (for the medical device 108 and/or for other devices associated with the HCP patient) to the HCP assistant system 102. Similarly, the population of additional HCPs and/or the population of additional patients may also provide corresponding HCP data, medical records, and device data to the HCP assistant system 102.

The HCP assistant system 102 collects, maintains, and processes the data collected from the HCP user, the HCP patient, the population of other HCPs, and the population of other patients. More specifically, the analytics engine 140 processes the input data and adaptively learns from the massive amounts of device and medical data that becomes available to the HCP assistant system 102. The analytics engine 140 generates appropriate output in a desired format that provides the HCP user with ideal and accurate therapy recommendations, suggested treatment plans, diagnostic information, suggested medications, and other helpful guidance that can be communicated to the HCP patient. In this regard, the generated content may include or be realized as one or more of the following, without limitation: a therapy adjustment recommendation; a patient insight message that is intended for delivery (e.g., queued or scheduled for delivery) to a patient; an HCP insight message that is intended for delivery to the HCP customer; and patient appointment information, which may include a prioritized list of topics to be addressed during an appointment with the HCP customer and a patient, and which may include appointment scheduling data, patient contact information, and the like.

In certain implementations, the HCP assistant system 102 sends reminders and relevant content to the HCP patient based on past behavior and live events measured on the patient's medical device and/or other patient devices. The chatbot feature of the HCP assistant system 102 can be configured and programmed to support real-time chat communication between the HCP patient, the HCP user, and the chatbot if so desired.

The following is an example of an output message generated by the HCP assistant system 102, where the intended recipient is the HCP user: “Your patient appears to have poor glucose control after late night snacks. We have sent her the attached recommendations based on your past therapy adjustments and preferences. Would you like to schedule an appointment with this patient?”

The following is another example of an output message intended for the HCP user: “Your patient is experiencing glucose variability after the delivery of large insulin boluses. Our data indicates that the patient's insulin sensitivity setting should be changed to X.”

The following is yet another example of an output message intended for the HCP user: “You have a few patients who struggle with glucose control in the morning. Follow the link below to see what other doctors have tried with similar patients, and to review what the resulting outcomes were.”

The following is an example of an output message generated by the HCP assistant system 102, where the intended recipient is the HCP patient: “It looks like you are over correcting for low glucose events. See the attached content for best practices. A copy of this message has been sent to your physician.”

The following is another example of an output message intended for the HCP patient: “You have an appointment tomorrow. Can you please confirm the appointment, and upload your device data?”

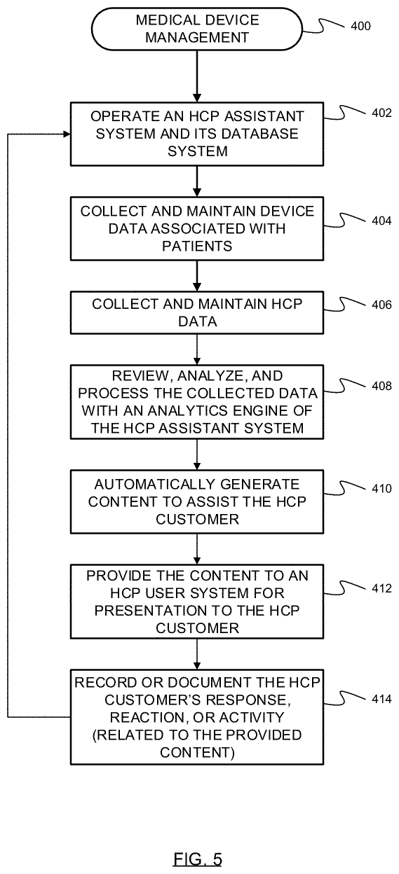

FIG. 5 is a flow chart that illustrates an exemplary embodiment of a medical device management process 400. The various tasks performed in connection with the process 400 may be performed by software, hardware, firmware, or any combination thereof. For illustrative purposes, the following description of the process 400 may refer to elements mentioned above in connection with FIGS. 1-4. It should be appreciated that an implementation of the process 400 may include any number of additional or alternative tasks, the tasks shown in FIG. 5 need not be performed in the illustrated order, and the process 400 may be incorporated into a more comprehensive procedure or process having additional functionality not described in detail herein. Moreover, one or more of the tasks shown in FIG. 5 could be omitted from an embodiment of the process 400 as long as the intended overall functionality remains intact.

The illustrated embodiment of the process 400 operates the HCP assistant system and its associated database system in an ongoing manner to support the HCP systems, the patient systems, and the medical devices in the manner described above (task 402). More specifically, the HCP assistant system and its database system are controlled and operated to receive, collect, and store/maintain the relevant input data that is used by the analytics engine of the HCP assistant system. To this end, the process 400 collects and maintains medical device user data, which can be associated with a plurality of different patients under the care of a plurality of different HCPs, and which can include data that is associated with the operation of medical devices and user devices associated with the different patients (task 404). The process 400 also collects and maintains HCP data, which can be associated with a plurality of different HCPs, and which can include data that is associated or otherwise related to the treatment of patients by the different HCPs (task 406).

This description continues in the context of an exemplary use case where a particular HCP (referred to here as the HCP customer) accesses or operates the HCP assistant system to view information related to patients under that particular HCP customer. Accordingly, the process 400 continues by reviewing, analyzing, and processing the collected medical device user data and the collected HCP data (task 408). As explained above, the analytics engine of the HCP assistant system processes the collected data using machine learning, artificial intelligence, and/or other advanced data processing algorithms, and the HCP assistant system automatically generates appropriate content to assist the HCP customer in managing treatment of his or her patients (task 410). The content is generated based on the review, analysis, and processing of the collected medical device user data and/or the collected HCP data. The generated content is formatted and arranged in a suitable manner such that it can be provided to the HCP user system for presentation to the HCP customer (task 412). For example, the generated content can be provided in the form of an HTML document for presentation as a web page in a web browser running on the HCP user system. Other output formats, representations, visualizations, and arrangements can also be generated by the HCP assistant system if so desired.

This description assumes that the HCP customer reviews the generated content and takes appropriate action in response to the reviewed content, wherein the action taken by the HCP customer can be documented or recorded. Accordingly, the process 400 may continue by recording or documenting the HCP customer's response, reaction, or activity (task 414). This information can be fed back to the HCP assistant system for purposes of adaptively updating the analytics engine to the extent needed. Such feedback enables the HCP assistant system to dynamically adjust its output going forward in a way that contemplates the past behavior of the HCP customer to improve the relevance and accuracy of future results and output provided to that HCP customer.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A medical device system comprising:
a medical device to provide therapy to a patient under the care of a health care professional (HCP) customer, the medical device comprising a processor device and a communication module that facilitate data communication between the medical device and at least one other component of the medical device system;
a computer-implemented HCP assistant system, the HCP assistant system comprising a processor device and a communication module that facilitate data communication between the HCP assistant system and at least one other component of the medical device system;
a database system, associated with the HCP assistant system, to collect and maintain medical device user data, and to collect and maintain HCP data, the medical device user data including data associated with the patient, and the HCP data including data associated with the HCP customer; and
a computer-implemented HCP user system, the HCP user system comprising a processor device and a communication module that facilitate data communication between the HCP user system and at least one other component of the medical device system;
the HCP assistant system operative to:
review the collected medical device user data and the collected HCP data;
automatically generate content to assist the HCP customer in managing treatment of the patient, based on the review of the collected medical device user data and the collected HCP data; and
provide the generated content to the HCP user system for presentation to the HCP customer.

2. The medical device system of claim 1, wherein the medical device comprises an insulin infusion device.

3. The medical device system of claim 2, wherein the collected medical device user data comprises at least some of: carbohydrate amount; bolus information; insulin to carbohydrate ratio; insulin sensitivity factor; active insulin amount; time of day; basal rate; temporary basal use; consecutive boluses; insulin suspension; reservoir rewind time; reservoir priming time; pump alarms and associated alarm times; sensor alerts and associated alert times; blood glucose readings and associated measurement times; user demographic information; meal times and corresponding meal content; exercise times and corresponding exercise content or type; medication type, dosage, and time; sleep time and quality; stress time; and electronic medical records; medical lab test data.

4. The medical device system of claim 1, wherein the generated content comprises a therapy adjustment recommendation.

5. The medical device system of claim 4, wherein the HCP user system is operative to initiate automatic adjustment of the medical device in accordance with the therapy adjustment recommendation.

6. The medical device system of claim 1, wherein the generated content comprises a patient insight message intended for delivery to the patient.

7. The medical device system of claim 6, wherein the HCP assistant system is operative to deliver the patient insight message to the patient only after receiving a delivery confirmation from the HCP user system.

8. The medical device system of claim 6, wherein the HCP assistant system is operative to deliver the patient insight message to the patient in accordance with delivery rules configured by the HCP user system.

9. The medical device system of claim 1, wherein the generated content comprises an HCP insight message intended for delivery to the HCP customer.

10. The medical device system of claim 1, wherein the generated content comprises patient appointment information including a prioritized list of topics to be addressed during an appointment with the HCP customer.

11. The medical device system of claim 1, wherein the medical device user data comprises data associated with a population of patients that includes the patient.

12. The medical device system of claim 1, wherein the HCP data comprises data associated with a population of HCPs that includes the HCP customer.

13. The medical device system of claim 1, wherein the collected HCP data comprises at least some of: patient medical records; patient appointment data; patient diagnostic data; treatment plans, therapy recommendations; patient medication data; patient examination notes; medical lab test data; electronic medical records; mobile device data; therapy-related notes, recommendations, and observations; and patient goals and objectives.

14. A computer-implemented health care provider (HCP) assistant system comprising:
at least one processor device; and
a non-transitory processor-readable medium operatively associated with the at least one processor device, the processor-readable medium comprising executable instructions configurable to cause the at least one processor device to perform a method comprising:
collecting and maintaining medical device user data that includes data associated with a patient under the care of an HCP customer;
collecting and maintaining HCP data that includes data associated with the HCP customer;
reviewing the collected medical device user data and the collected HCP data;
automatically generating content to assist the HCP customer in managing treatment of the patient, based on the review of the collected medical device user data and the collected HCP data; and
providing the generated content to an HCP user system for presentation to the HCP customer.

15. The HCP assistant system of claim 14, wherein the generated content comprises a therapy adjustment recommendation.

16. The HCP assistant system of claim 14, wherein the generated content comprises a patient insight message intended for delivery to the patient.

17. The HCP assistant system of claim 14, wherein the generated content comprises an HCP insight message intended for delivery to the HCP customer.

18. The HCP assistant system of claim 14, wherein the generated content comprises patient appointment information including a prioritized list of topics to be addressed during an appointment with the HCP customer.

19. A method of managing use of a medical device that provides therapy to a patient under the care of a health care professional (HCP) customer, the method comprising:
operating a database system associated with a computer-implemented HCP assistant system, the HCP assistant system comprising a processor device and a communication module that facilitate data communication between the HCP assistant system and the medical device;
collecting and maintaining medical device user data at the database system, the medical device user data including data associated with the patient and further including data associated with operation of the medical device;
collecting and maintaining HCP data at the database system, the HCP data including data associated with the HCP customer and further including data associated with treatment of the patient by the HCP customer;
reviewing the collected medical device user data and the collected HCP data;
automatically generating content to assist the HCP customer in managing treatment of the patient, based on the review of the collected medical device user data and the collected HCP data; and
providing the generated content to an HCP user system for presentation to the HCP customer.

20. The method of claim 19, wherein the generated content comprises:
a patient insight message intended for delivery to the patient;
an HCP insight message intended for delivery to the HCP customer; or
patient appointment information including a prioritized list of topics to be addressed during an appointment with the HCP customer.
